(19) United States
(12) Plant Patent Application Publication
(10) Pub. No.: US 2016/0302341 P1
(43) Pub. Date: Oct. 13, 2016

(54) Convallaria Plant Named 'Fragol'

(50) ;

(71) Applicant: Anthony Brooks, Elton nr. Ludlow (GB)

(72) Inventor: Anthony Brooks, Elton nr. Ludlow (GB)

(21) Appl. No.: 14/545,197

(22) Filed: Apr. 7, 2015

(51) Int. Cl. A01H 5/00

(52) U.S. Cl. PLT/314

(57) ABSTRACT

A new and distinct cultivar of Convallaria plant named ‘Fragol’, characterized by its upright to spreading plant habit; moderately vigorous to vigorous growth habit; yellow green-colored leaves; freely and early flowering habit; relatively large campanulate flowers that are strongly fragrant and white in color; and good garden performance.

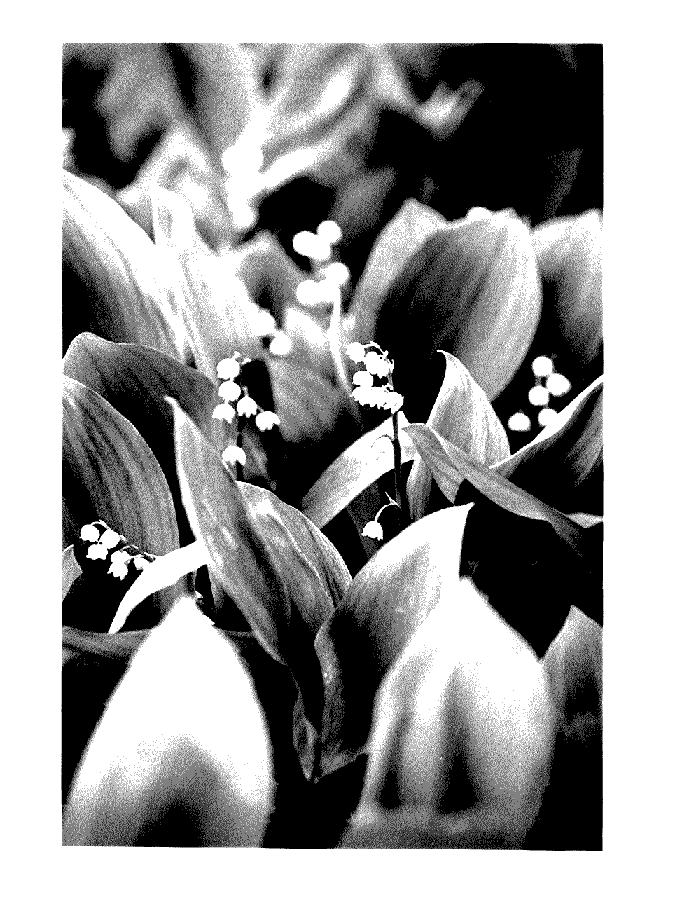

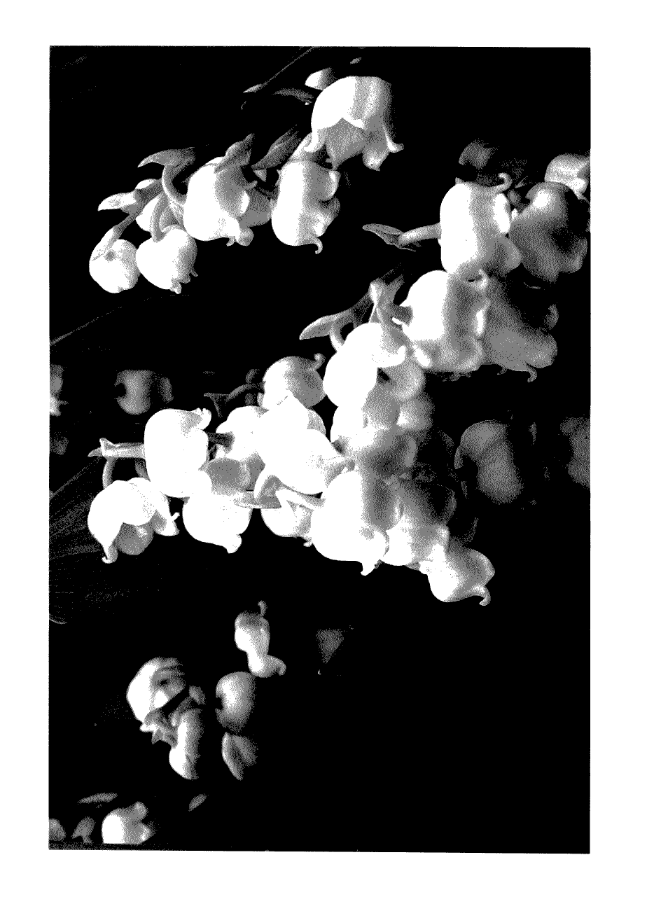

### BOTANICAL DESIGNATION

Convallaria majalis

### CULTIVAR DENOMINATION

‘FRAGOL’

### BACKGROUND OF THE INVENTION

The present invention relates to a new and distinct cultivar of Convallaria plant, botanically known as Convallaria majalis, commonly referred to as Lily-Of-The-Valley, and hereinafter referred to by the cultivar name ‘Fragol’.

The new Convallaria plant is a whole plant mutation of Convallaria majalis ‘Hardwick Hall’, not patented. The new Convallaria plant was discovered and selected by the Inventor as a single flowering plant from within a population of plants of ‘Hardwick Hall’ grown in an outdoor nursery environment in Shropshire, United Kingdom during the spring of 1996.

Asexual reproduction of the new Convallaria plant by divisions in a controlled greenhouse environment in Shropshire, United Kingdom since June, 1996 has shown that the unique features of this new Convallaria plant are stable and reproduced true to type in successive generations.

### SUMMARY OF THE INVENTION

Plants of the new Convallaria have not been observed under all possible combinations of environmental conditions and cultural practices. The phenotype may vary somewhat with variations in environmental conditions such as temperature and light intensity without, however, any variance in genotype.

The following traits have been repeatedly observed and are determined to be the unique characteristics of ‘Fragol’. These characteristics in combination distinguish ‘Fragol’ as a new and distinct Convallaria plant:

1. Upright to spreading plant habit.

2. Moderately vigorous to vigorous growth habit.

3. Yellow green-colored leaves.

4. Freely and early flowering habit.

5. Relatively large campanulate flowers that are strongly fragrant and white in color.

6. Good garden performance.

Plants of the new Convallaria differ primarily from the mutation parent, ‘Hardwick Hall’, in the following characteristics:

1. Plants of the new Convallaria are smaller than and not as vigorous as plants of ‘Hardwick Hall’.

2. Plants of the new Convallaria and ‘Hardwick Hall’ differ in leaf color as leaves of plants of ‘Hardwick Hall’ are pale green in color with a narrow creamy yellow-colored border.

3. Plants of the new Convallaria are more freely flowering than plants of ‘Hardwick Hall’.

4. Flowers of plants of the new Convallaria are larger and more fragrant than flowers of plants of ‘Hardwick Hall’.

5. Plants of the new Convallaria have shorter peduncles than plants of ‘Hardwick Hall’.

Plants of the new Convallaria can be compared to plants of Convallaria majalis ‘Albrostriata’, not patented. In side-by-side comparisons conducted in Shropshire, United Kingdom, plants of the new Convallaria differ primarily from plants of ‘Albrostriata’ in leaf color as plants of ‘Albrostriata’ have green-colored leaves with fine creamy yellow-colored longitudinal stripes.

Plants of the new Convallaria can also be compared to plants of Convallaria majalis ‘Golden Jubilee’, not patented. In side-by-side comparisons conducted in Shropshire, United Kingdom, plants of the new Convallaria differ primarily from plants of ‘Golden Jubilee’ in leaf color as plants of ‘Golden Jubilee’ have light yellow green-colored leaves. In addition, plants of the new Convallaria have broader leaves than plants of ‘Golden Jubilee’.

### BRIEF DESCRIPTION OF THE PHOTOGRAPHS

The accompanying colored photographs illustrate the overall appearance of the new Convallaria plant showing the colors as true as it is reasonably possible to obtain in colored reproductions of this type. Colors in the photographs may differ slightly from the color values cited in the detailed botanical description which accurately describe the colors of the new Convallaria plant. The photograph on the first sheet comprises a side perspective view of a typical flowering plant of ‘Fragol’ grown in a container. The photograph on the second sheet is a close-up view of a typical flowering plant of ‘Fragol’.

### DETAILED BOTANICAL DESCRIPTION

The aforementioned photographs and following observations, measurements and values describe plants grown during the spring and summer in five-liter containers and ground beds in an outdoor nursery in Bressingham, Diss, Norfolk, United Kingdom and under cultural practices typical of commercial Convallaria production. During the production of the plants, day temperatures ranged from 18° C. to 22° C. and night temperatures ranged from 6° C. to 15° C. Plants were three years old when the photographs and the description were taken. In the following description, color references are made to The Royal Horticultural Society Colour Chart, 2000 Edition, except where general terms of ordinary dictionary significance are used.
Botanical classification: Convallaria majalis ‘Fragol’.
Parentage: Whole plant mutation of Convallaria majalis ‘Hardwick Hall’, not patented.
Propagation:
   Type.—By divisions.
   Time to initiate roots, summer.—About one month at soil temperatures about 16° C. to 20°.
   Time to produce a rooted young plant, summer.—About three months at temperatures about 16° C. to 20° C.
   Root description.—Medium in thickness, fleshy main roots and fibrous lateral roots; white, close to 158B, in color.
   Rooting habit.—Freely branching; medium density.
   Rhizomes.—Length: About 30 cm. Diameter: About 30 cm. Texture: Smooth, glabrous. Color: Close to 159C.
Plant description:
   Plant and growth habit.—Herbaceous perennial; upright and spreading plant habit with flowers held above the foliar plane; moderately vigorous to vigorous growth habit.
   Plant height, soil level to top of flowers.—About 20 cm.
   Plant diameter (area of spread).—About 75 cm.
   Stem aspect.—Mostly upright.
   Stem strength.—Strong.
   Stem texture.—Smooth, glabrous.
   Internode length.—About 2 cm.
   Color.—Close to 161B; new shoots with reddish flush.
Leaf description:
   Arrangement.—Opposite; sessile.
   Length.—About 15 cm.
   Width.—About 10 cm.
   Shape.—Ovate.
   Apex.—Apiculate.
   Base.—Cuneate.
   Margin.—Entire.
   Texture, upper surface.—Smooth, glabrous.
   Texture, lower surface.—Slightly ridged, glabrous.
   Venation pattern.—Camptodrome.
   Color.—Developing leaves, upper surface: Close to 150C. Developing leaves, lower surface: Close to 150D. Fully developed leaves, upper surface: Close to 149C; venation, close to 145A. Fully developed leaves, lower surface: Close to 149D; venation, close to 145B.
Flower description:
   Flower shape and habit.—Single campanulate flowers arranged on terminal racemes; freely and uniform flowering habit with about 8 to 15 flowers per raceme and about 200 flowers developing per plant; flowers nodding.
   Fragrance.—Strongly fragrant; pleasant, sweet.
   Natural flowering season.—Early flowering habit, plants begin flowering about 56 days after planting; in the garden, plants flower continuously during May and June in the United Kingdom.
   Flower longevity on the plant.—About one month; flowers not persistent.
   Inflorescence height.—About 20 cm to 22 cm.
   Inflorescence diameter.—About 6 cm to 7 cm.
   Flower buds.—Length: About 5 mm. Diameter: About 5 mm. Shape: Rounded. Color: Close to 154D.
   Flower diameter.—About 1.5 cm.
   Flower depth (height).—About 2 cm.
   Petals.—Quantity and arrangement: Six fused into a campanulate structure. Length: About 2 cm. Width: About 5 mm. Apex: Acute; reflexed. Margin: Entire. Texture, upper and lower surfaces: Smooth, glabrous; waxy. Color: When opening, upper and lower surfaces: Close to 155B. Fully opened, upper and lower surfaces: Close to 155B; color becoming closer to 156C with development.
   Flower bracts.—Quantity and arrangement: One subtending the flower. Length: About 2 cm. Width: About 5 mm. Shape: Obovate. Apex: Obtuse. Base: Cuneate. Margin: Entire. Texture, upper and lower surfaces: Smooth, glabrous. Color, upper and lower surfaces: Close to 154D.
   Peduncles.—Length: About 22 cm. Diameter: About 3 mm. Aspect: Upright to slightly nodding. Strength: Strong, flexible. Texture: Smooth, glabrous. Color: Close to 154D.
   Pedicels.—Length: About 5 mm. Diameter: About 2 mm. Aspect: Nodding. Strength: Strong, flexible. Texture: Smooth, glabrous. Color: Close to 154D.
   Reproductive organs.—Stamens: Quantity per flower: About six. Filament length: About 0.4 mm. Filament color: Yellow green. Anther shape: Elliptical. Anther length: About 0.3 mm. Anther color: Yellow green. Pollen amount: Scarce. Pollen color: Very light yellow brown. Pistils: Quantity per flower: One. Pistil length: About 0.4 mm. Stigma shape: Tubular. Stigma color: Yellow green. Style length: About 0.4 mm. Style color: Yellow green. Ovary color: Yellowish.
   Seeds and fruits.—Seed and fruit development have not been observed on plants of the new Convallaria.
Garden performance: Plants of the new Convallaria have been observed to have good garden performance and to tolerate rain, wind and temperatures ranging from −20° C. to 25° C.
Pathogen & pest resistance: Plants of the new Convallaria have been observed to be tolerant to Rust pathogens. Plants of the new Convallaria have not been observed to be resistant to pests and other pathogens common to Convallaria plants.

It is claimed:

1. A new and distinct Convallaria plant named ‘Fragol’ as illustrated and described.
